(19) United States
(12) Patent Application Publication
(10) Pub. No.: US 2018/0360758 A1
(43) Pub. Date: Dec. 20, 2018

(54) NANOLIPOSOMES COMPRISING CORTICOSTEROID AS MEDICAMENTS AND METHODS TO PREPARE THEM

(71) Applicant: NANYANG TECHNOLOGICAL UNIVERSITY, Singapore (SG)

(72) Inventors: Subramanian VENKATRAMAN, Singapore (SG); Jayaganesh V. NATARAJAN, Singapore (SG); Anastasia DARWITAN, Singapore (SG)

(21) Appl. No.: 16/063,062

(22) PCT Filed: Nov. 28, 2016
(86) PCT No.: PCT/SG2016/050584

§ 371 Date: Jun. 15, 2018

(30) Foreign Application Priority Data
Dec. 17, 2015 (SG) 10201510370T

(51) Int. Cl. A61K 9/127; A61K 31/57

(52) U.S. Cl. CPC A61K 9/1271; A61K 31/57

(57) ABSTRACT

Nanoliposomes comprising at least one outer lipid bilayer and at least one corticosteroid encapsulated by the at least one lipid bilayer are provided. Uses of the nanoliposomes as a medicament and in the treatment of a cardiovascular disease are also provided. Further, methods of preparing the nanoliposomes are additionally provided.

### FIELD OF THE INVENTION

The present invention lies in the field of bio-pharmaceutical chemistry and relates to a nanoliposome comprising at least one outer lipid bilayer and at least one corticosteroid encapsulated by the at least one lipid bilayer. The present invention also relates to the use of the nanoliposome of the invention for use as a medicament and for use in the treatment of a cardiovascular disease. Further, the invention is directed to a method to prepare the nanoliposome of the invention.

### BACKGROUND OF THE INVENTION

Atherosclerosis is a systemic vascular disease commonly grouped under the broad generic term of cardiovascular diseases. Cardiovascular diseases accounts for approximately 30% of overall mortality worldwide and is an important medical problem that needs viable treatment solutions. Atherosclerosis is considered to be a global socio-economic problem with substantial financial burden due to changing demographics and urban lifestyle and this problem exists regardless of geography, gender or ethnicity. Current treatment strategies for atherosclerosis include the use of drugs that lower the levels of triglycerides, LDL-cholesterol levels, reduce blood pressure and platelet aggregation. However, limitations with current treatment strategies is that the activity of these drugs is not limited to the site of action (i.e. in atherosclerotic lesions) and additionally, these drugs show limited therapeutic efficacy due to shorter plasma half-life and increased plasma clearance. Most drugs show moderate to severe systemic-side effects such as uncontrolled bleeding with antiplatelet drugs and reduced efficacy for fibrinolytic drugs such as tissue plasminogen activator (tPA) and streptokinase due to rapid degradation and wash out. Recent strategies include the use of anti-inflammatory drugs for the treatment of atherosclerosis, in lieu of evidence that inflammatory process is a key factor for progression of the disease and pathogenesis.

One of the strategies to treat diffuse (middle to late stage) atherosclerotic plaques is the targeting of monocytes and macrophages with anti-inflammatory drug-loaded nanoliposomes. Innate immunity is an inherent response from the human body defense mechanism which plays a major role in vascular repair. Once the injury is inflicted, there is an immediate recruitment of both macrophages and monocytes to the injury site mimicking a normal wound healing process. The recruited macrophages then activate and stimulate a series of cell signaling mechanisms that lead to cellular changes such as expression of growth factors and cytokines, proliferation and migration of smooth muscle cells that eventually result in atherosclerotic lesions and formation of “foam” cells. With monocytes and macrophages as the primary target, anti-inflammatory drug loaded nanoliposomes would be able to minimize and eliminate the uncontrolled pro-inflammatory response at the sub-endothelial space thus preventing progression of the disease.

Therefore, there is a need in the art to develop nanoliposomes that target monocytes and macrophages to atherosclerotic plaques.

### SUMMARY OF THE INVENTION

It is an object of the present invention to meet the above need by providing nanoliposome comprising at least one outer lipid bilayer and at least one corticosteroid encapsulated by the at least one lipid bilayer as described herein. The present inventors have developed sustained release nanoliposomes encapsulating corticosteroids that could be a potential effective treatment strategy for atherosclerosis by systemic administration. Many existing technologies do not take into account the need for sustained drug delivery for atherosclerosis which could be a potential drawback for prolonged therapeutic efficacy. This invention is based on the hypothesis that upon injection of the nanoliposomes systemically, the drug loaded nanoliposomes (either by passive or active targeting) will transport to the site of action i.e. in atherosclerotic lesions and release the drug over a long period of time (days to months). This would drastically improve patient compliance and as well minimize side effects associated with the need of frequent injections to halt the progression of the disease.

In the present application, the present inventors were able to achieve high loading concentrations of corticosteroids (up to 1.2 mg/ml) into liposomes using passive loading. In addition, controlled and sustained release of various corticosteroid drugs (e.g. fluocinolone acetonide and triamcinolone acetonide) from nanoliposomes has been demonstrated by an in vitro dialysis. The release of the drug was sustained up to forty days in in-vitro dialysis.

Further, high loading concentrations of corticosteroids into liposomes have been demonstrated. These high loading concentrations may dependent on the ratio of the drug to lipid amounts used for preparing the nanoliposome of the invention.

In a first aspect, the present invention is thus directed to a nanoliposome comprising at least one outer lipid bilayer and at least one corticosteroid encapsulated by the at least one lipid bilayer.

In a second aspect, the present invention is directed to the nanoliposome of the present invention for use as a medicament.

In a further aspect, the invention relates to the nanoliposome of the invention for use in the treatment of a cardiovascular disease.

Finally, the present invention relates in a fourth aspect to a method to prepare the nanoliposome of the invention, comprising: a) providing a composition comprising the lipids forming the at least one lipid bilayer and a solvent; b) adding the at least one corticosteroid to the composition of step a); and c) removing the solvent to prepare the nanoliposome of the invention.

### BRIEF DESCRIPTION OF THE DRAWINGS

The invention will be better understood with reference to the detailed description when considered in conjunction with the non-limiting examples and the accompanying drawings.

FIG. 1 shows the size measurement of liposomes during storage at 4° C. and after in-vitro drug release study in PBS buffer (pH 7.4) at 37° C.

FIG. 2 shows an in-vitro drug release study of fluocinolone acetonide from DPPC liposomes (drug/lipid mole ratio of 0.135) and DPPC (95%), DSPE-PEG 2K (5%) liposomes (drug/lipid mole ratio of 0.151). (a) Cumulative fluocinolone acetonide release (%) plotted against time (days) and (b) daily amount of fluocinolone acetonide release (μg) from 1 mL liposomal solution dialyzed against 40 mL PBS pH 7.4.

FIG. 3 shows the summary of initial drug/lipid (D/L), final D/L ratios, final drug concentration, partition coefficient (PC), % encapsulation efficiency (EE), % loading efficiency (LE), and average size after extrusion for various saturated plain and pegylated nanoliposomes.

FIG. 4 shows an in-vitro drug release study of fluocinolone acetonide from DPPC liposomes (drug/lipid mole ratio of 0.114). (a) Cumulative fluocinolone acetonide release (%) plotted against time (days) and (b) daily amount of fluocinolone acetonide release (μg) from 1 mL liposomal solution dialyzed against 40 mL PBS pH 7.4.

FIG. 5 shows an in-vitro drug release study of fluocinolone acetonide from DPPC (95%), DSPE-PEG 2K (5%) liposomes (drug/lipid mole ratio of 0.117). (a) Cumulative fluocinolone acetonide release (%) plotted against time (days) and (b) daily amount of fluocinolone acetonide release (μg) from 1 mL liposomal solution dialyzed against 40 mL PBS pH 7.4.

FIG. 6 shows the summary of initial drug/lipid (D/L), final D/L ratios, final drug concentration, partition coefficient (PC), % encapsulation efficiency (EE), % loading efficiency (LE), and average size after extrusion for pegylated nanoliposomes.

FIG. 7 shows an in-vitro drug release study of triamcinolone acetonide from DPPC (95%), DSPE-PEG 2K (5%) liposomes (drug/lipid mole ratio of 0.096). (a) Cumulative triamcinolone acetonide release (%) plotted against time (days) and (b) daily amount of fluocinolone acetonide release (μg) from 1 mL liposomal solution dialyzed against 40 mL PBS pH 7.4.

FIG. 8 shows the summary of initial drug/lipid (D/L), final D/L ratios, partition coefficient (PC), % encapsulation efficiency (EE), % loading Efficiency (LE), final drug concentration and average size after extrusion for nanoliposomes comprising of sphingomyelin.

FIG. 9 shows an in-vitro drug release study of fluocinolone acetonide from sphingomyelin liposomes (drug/lipid mole ratio of 0.087). (a) Cumulative fluocinolone acetonide release (%) plotted against time (days) and (b) daily amount of fluocinolone acetonide release (μg) from 1 mL liposomal solution dialyzed against 40 mL PBS pH 7.4.

FIG. 10 shows the summary of initial drug/lipid (D/L), final D/L ratios, partition coefficient (PC), % encapsulation efficiency (EE), % loading efficiency (LE), final drug concentration, average size and zeta potential after extrusion for nanoliposomes comprising of charged lipids.

FIG. 11 shows an in-vitro drug release study of fluocinolone acetonide from mixture of DPPC and DOTAP liposomes (50% mole ratio) (drug/lipid mole ratio of 0.157). (a) Cumulative fluocinolone acetonide release (%) plotted against time (days) and (b) daily amount of fluocinolone acetonide release (μg) from 1 mL liposomal solution dialyzed against 40 mL PBS pH 7.4.

FIG. 12 shows an in-vitro drug release study of fluocinolone acetonide from mixture of DMPC and DOTAP liposomes (50% mole ratio) (drug/lipid mole ratio of 0.157). (a) Cumulative fluocinolone acetonide release (%) plotted against time (days) and (b) daily amount of fluocinolone acetonide release (mg) from 1 mL liposomal solution dialyzed against 40 mL PBS pH 7.4.

FIG. 13 shows the summary of initial drug/lipid (D/L), final D/L ratios, partition coefficient (PC), % encapsulation efficiency (EE), % loading efficiency (LE) and final drug concentration after extrusion for nanoliposomes comprising of DPPC with lipid concentration of 36 mM.

FIG. 14 shows an in-vitro drug release study of fluocinolone acetonide from mixture of DPPC liposomes (lipid concentration 36 mM) (drug/lipid mole ratio of 0.116). (a) Cumulative fluocinolone acetonide release (%) plotted against time (days) and (b) daily amount of fluocinolone acetonide release (μg) from 1 mL liposomal solution dialyzed against 40 mL PBS pH 7.4.

### DETAILED DESCRIPTION OF THE INVENTION

The present inventors surprisingly found that nanoliposomes of the present invention have the capacity for sustained release of corticosteroids (up to 40 days). Further, under storage conditions (4° C.) said nanoliposomes are stable for over three months. Moreover, the high loading concentrations of corticosteroids into liposomes have been demonstrated. These high loading concentrations may dependent on the ratio of the drug to lipid amounts used for preparing the nanoliposome of the invention.

Therefore, in a first aspect, the present invention is thus directed to a nanoliposome comprising at least one outer lipid bilayer and at least one corticosteroid encapsulated by the at least one lipid bilayer.

The term “liposome”, as used herein, refers to an artificially prepared vesicle composed of a lipid bilayer. A liposome may be classified as a unilamellar vesicle or a multivesicular vesicle. In preferred embodiments of the present invention, the liposome is a unilamellar vesicle.

Liposomes consist of at least one lipid bilayer membrane enclosing an aqueous internal compartment. Liposomes may be characterized by membrane type and by size. Small unilamellar vesicles (SUVs) may have a single membrane and have a diameter in a range of about 20 nm to about 50 nm. Large unilamellar vesicles (LUVs) may have a diameter of about 50 nm or greater. Oligolamellar large vesicles and multilamellar vesicles may have multiple, usually concentric, membrane layers and have a diameter of about 100 nm or greater. Liposomes with several non-concentric membranes, i.e., several smaller vesicles contained within in a large vesicle, are termed multivesicular vesicles.

The liposome may be a non-stimulus or stimulus-sensitive liposome (i.e., sensitive to one or more stimuli), and the stimulus-sensitive liposome may control release of materials that are encapsulated therein. As used herein, “sensitive” to stimuli refers to the ability of a liposome to release its contents in response to exposure to one or more stimuli or the like, or to disintegrate in response to one or more stimuli or the like. Examples of the stimulus-sensitive liposome include a temperature-sensitive liposome, a pH-sensitive liposome, a chemical-sensitive liposome, a radiation-sensitive liposome, an ultrasound-sensitive liposome, or any combination thereof. The temperature-sensitive liposome, the pH-sensitive liposome, the chemical-sensitive liposome, the radiation-sensitive liposome, and the ultrasound-sensitive liposome may release materials that are contained therein at a certain temperature or temperature range, a certain pH or pH range, the presence of chemical substance, radiation conditions, and/or ultrasound conditions. The temperature may be, for example, in a range of about 25° C. to about 70° C., about 25° C. to about 65° C., about 25° C. to about 60° C., about 25° C. to about 55° C., about 25° C. to about 50° C., about 30° C. to about 50° C., about 35° C. to about 50° C., or about 37° C. (body temperature) to about 50° C. The pH may be greater than, equal to, or less than about 5.5, which is the pH of saline solution. As used herein “ultrasound” refers to a wave with a frequency greater than an audio frequency ranging from about 16 Hz to about 20 kHz. The ultrasound may be high intensity focused ultrasound (HIFU), and HIFU involves high-intensity ultrasound energies in one place to create a concentrated focus.

The term “nano”, as used herein, describes a nanosized material, for example a single liposome, which is less than 150 nanometers, preferably less than 100 nanometers.

Therefore, the term “nanoliposomes”, as used herein, refers to liposomes that having the above referred properties and having a diameter of 10 nm to 1000 nm, preferably 50 nm to 150 nm. Methods to prepare such liposomes include extrusion methods, sonication methods and the Mozafari method (Blume, G; Cevc, G (1990). “Liposomes for the sustained drug release in vivo”. Biochimica et Biophysica Acta. 1029 (1): 92-97). It is well-known to the skilled person that different parameter of the preparation process, such as time and intensity of extrusion or sonication, influence to diameter size of the resulting liposomes.

The terms “at least one” and “plurality”, as interchangeably used herein, relate to one or more, in particular 1, 2, 3, 4, 5, 6, 7, 8, 9, 10, 50, 100, 500, 1000 or more.

The term “lipid bilayer”, as used herein, refers to a membrane made of two layers of lipid molecules. The lipid bilayer may have a similar thickness as that of a naturally existing bilayer, such as a cell membrane, a nuclear membrane, and a virus envelope. For example, the lipid bilayer may have a thickness of about 10 nm or less, for example, in a range of about 1 nm to about 9 nm, about 2 nm to about 8 nm, about 2 nm to about 6 nm, about 2 nm to about 4 nm, or about 2.5 nm to about 3.5 nm. The lipid bilayer is a barrier that keeps ions, proteins, and other molecules in an area, and/or prevents them from diffusing into other areas. The “lipid molecules” or “lipids” forming the lipid bilayer may be a molecule including a hydrophilic head and hydrophobic tails. The lipid molecule may have 14 to 50 carbon atoms.

In various embodiments, the lipid bilayer may be phospholipid, a lipid conjugated to polyethylene glycol (PEG), cholesterol, elastin-like polypeptide, a sphingolipid or any combination thereof.

As used herein “phospholipid” refers to a compound lipid containing phosphate ester within a molecule, and is a main component of biological membranes, such as cell membranes, endoplasmic reticulum, mitochondria, and myelin sheath around nerve fibers. The phospholipid includes a hydrophilic head and two hydrophobic tails. When the phospholipids are exposed to water, they arrange themselves into a two-layered sheet (a bilayer) with all of their tails pointing toward the center of the sheet. The center of this bilayer contains almost no water and also excludes molecules such as sugars or salts that dissolve in water but not in oil. The phospholipid may include phosphatidic acid, phosphatidylethanolamine, phosphatidylcholine, phosphatidylserine, phosphatidylinositol, phosphosphingolipid, or any combination thereof. Phosphatidylcholine or phosphocholine (PC), as interchangeably used herein, may include choline as a head group and glycerophosphoric acid as a tail, wherein glycerophosphoric acid may be saturated fatty acid or unsaturated fatty acid and have 14 to 50 carbon atoms. Examples of the PC include 1,2-dipalmitoyl-sn-glycero-3-phosphocholine (DPPC), 1,2-distearoyl-sn-glycero-3-phosphocholine (DSPC), egg PC, soy bean PC, and any combination thereof.

In some embodiments, the lipid may be conjugated to poly(ethylene glycol) (PEG). The PEG-lipid conjugate may be, for example, PEGylated phosphatidylethanolamine (PE)-PEG. The PE may be saturated fatty acid, unsaturated fatty acid, mixed acyl chain, lysophosphatidylethanolamine, or any combination thereof. The lipid conjugated to PEG may, be for example, 1,2-distearoylphosphatidylethanolamine-methyl-polyethylene glycol (DSPE-PEG).

The term “cholesterol”, as used herein, refers any steroid compounds. Cholesterol also includes a cholesterol derivative, and examples thereof include sitosterol, ergosterol, stigmasterol, 4,22-stigmastadiene-3-on, stigmasterol acetate, lanosterol, cycloartenol, or any combination thereof. Cholesterol may enhance fluidity of a lipid bilayer and lower the permeability of the lipid bilayer.

The term “elastin-like polypeptide (ELP)”, as used herein, refers to one type of an amino acid polymer which undergoes conformation changes according to temperature. In some embodiments, ELP may be a polymer having “inverse phase transitioning behavior”. The term “inverse phase transitioning behavior” as used herein refers to a substance having solubility in an aqueous solution at temperature below an “inverse phase transition temperature (Tₜ)” or a substance having insolubility in an aqueous solution at temperature above Tₜ. As the temperature rises, the ELP may transition into a tightly folded aggregate having solubility that is significantly decreased from the highly soluble elongated chain. That is, such an inverse phase transition may be induced by ELP further including a 6-turn or distorted 6-structure depending on the temperature. The ELP may have, for example, a phase transition temperature in a range of about 10° C. to about 70° C., about 20° C. to about 70° C., about 30° C. to about 70° C., about 37° C. (body temperature) to about 70° C., about 39° C. to about 70° C., about 40° C. to about 70° C., about 50° C. to about 70° C., or about 50° C. to about 70° C.

A “sphingolipid”, as used herein, is any of a group of lipids that yields sphingosine or its derivatives upon hydrolysis. Non-limiting examples of sphingolipids include sphingomyelins and glycosphingolipids such as cerbrosides, gangliosides, and sulfatides. The term “sphingolipid” means a natural and synthetic substance comprising a long-chain base (LCB) (i.e. sphingoid base, a long-chain hydrocarbon material derived from d-erythro-2-amino-1,3-diol), generally comprising a polar head group, and may include reference to such compounds further comprising an amide-linked fatty acid, or to such compounds generally referred to as “lysosphingolipids” for the N-deacylated form from which the fatty acid chain bonded via an acid-amide bond to the amino group of the sphingoid has been eliminated. Preferred sphingolipids in aspects of the present invention are lysosphingolipids, most preferably sphingoid bases.

The term “sphingoid base”, as used herein, refers to long chain amino alcohols that may differ in length of the alkyl chain lengths and extend of branching. The most common long-chain bases in mammals are sphingosine, sphinganine and phytosphingosine.

In various embodiments of the present invention, lipids of the at least one lipid bilayer are selected from the group consisting of dipalmitoylphosphatidylcholin (DPPC), 1,2-distearoyl-sn-glycero-3-phosphoethanolamine (DSPE), sphingomyelin, N-[1-(2,3-Dioleoyloxy)propyl]-N,N,N-trimethylammonium methyl-sulfate (DOTAP), 1,2-dimyristoyl-sn-glycero-3-phosphocholine (DMPC), 1,2-dimyristoyl-sn-glycero-3-phospho-(1′-rac-glycerol) (DMPG), soy hydrogenated L-α-phosphatidylcholine (HSPC), cholesterol, 1,2-distearoyl-sn-glycero-3-phospho-(1′-rac-glycerol) (DSPG), 1,2-dioleoyl-sn-glycero-3-phosphocholine (DOPC), 1,2-dipalmitoyl-sn-glycero-3-phospho-(1′-rac-glycerol) (DPPG), 1,2-distearoyl-sn-glycero-3-phosphocholine (DSPC), phospholipids from hen's egg, soybean oil and polysorbate 80.

The term “corticosteroid”, as used herein, refers to any of a wide variety of drugs that are closely related to cortisol, a hormone which is naturally produced in the adrenal cortex. Corticosteroids are sub-divided into group A, B, C, D₁ and D₂. Group A includes the following compounds: hydrocortisone, hydrocortisone acetate, cortisone acetate, tixocortol pivalate, prednisolone, methylprednisolone, and prednisone. Group B includes the following compounds: triamcinolone acetonide, triamcinolone alcohol, mometasone, amcinonide, budesonide, desonide, fluocinonide, fluocinolone acetonide, and halcinonide. Group C includes the following compounds: betamethasone, betamethasone sodium phosphate, dexamethasone, dexamethasone sodium phosphate, and fluocortolone. Group D₁ includes the following compounds: Hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate. Group D₂ includes the following compounds: hydrocortisone-17-butyrate, hydrocortisone-17-aceponate, hydrocortisone-17-buteprate, ciclesonide and prednicarbate. In preferred embodiments, the corticosteroid is a group B corticosteroid. In other preferred embodiments, the corticosteroid is selected from the group consisting of triamcinolone acetonide, fluocinolone acetonide, triamcinolone alcohol, mometasone, amcinonide, budesonide, desonide, fluocinonide and halcinonide.

In preferred embodiments of the present invention, the ratio of the at least one corticosteroid and the lipids forming the at least one lipid bilayer is between 0.01 and 0.5, preferably between 0.1 and 0.3, and more preferably between 0.12 and 0.18. This ratio refers to the total amount of corticosteroids and lipids forming the lipid bilayer. The amount is measured by weight of the corticosteroid(s) and the lipid(s). Thus, for example, if the amount of 1 g of corticosteroid and the amounts of 1 g of lipid A and 1g of lipid B is used to prepare the nanoliposome of the invention, then the resulting drug/lipid ratio is 0.5 (1g of corticosteroid/[1 g lipid A+1 g lipid B]).

In other preferred embodiments, the size of the liposome is between 10 nm to 1000 nm, preferably between 50 nm to 150 nm. The term “size of the liposome”, as used herein, refers to the diameter of the most outer lipid bilayer of the liposome.

In various embodiments of the invention, the at least one lipid bilayer comprises at least two different types of lipids. Therefore, the lipid bilayer may be formed of a combination of DPPC/DSPE-PEG, DPPC/DOTAP or DMPC/DOTAP.

Also included within the scope of the present invention are embodiments wherein the lipids forming the at least one lipid bilayer are modified by polyethylene glycol (PEG) and/or the at least one lipid bilayer comprises non-coupled polyethylene glycol (PEG). The term “PEG”, as used herein, means a polyethylene glycol molecule. In its typical form, PEG is a linear polymer with terminal hydroxyl groups and has the formula HO—CH₂CH₂—(CH₂CH₂O)ₙ—CH₂CH₂—OH, where n is from about 8 to about 4000. Typically, n is not a discrete value but constitutes a range with approximately Gaussian distribution around an average value. The terminal hydrogen may be substituted with a capping group such as an alkyl or alkanol group. Preferably, PEG has at least one hydroxy group, more preferably it is a terminal hydroxy group. This hydroxy group is preferably attached to a linker moiety which can react with the lipid to form a covalent linkage. Numerous derivatives of PEG exist in the art. (See, e.g., U.S. Pat. Nos. 5,445,090; 5,900,461; 5,932,462; 6,436,386; 6,448,369; 6,437,025; 6,448,369; 6,495,659; 6,515,100 and 6,514,491 and Zalipsky, S. Bioconjugate Chem. 6:150-165, 1995). The PEG molecule free or covalently attached to the lipid forming the bilayer may be approximately 10,000, 20,000, 30,000, or 40,000 daltons average molecular weight. The PEG molecule is preferably 18,000 to 22,000 daltons. More preferably, it is 19,000 to 21,000 Daltons. Most preferably it is 20,000 to 21,000 daltons. It is even more preferably approximately 20,000 daltons. PEGylation reagents may be linear or branched molecules and may be present singularly or in tandem. The term “PEGylation” or “modified with PEG”, as interchangeably used herein, means the covalent attachment of one or more PEG molecules, as described above, to a lipid molecule forming the lipid bilayer of nanoliposome of the invention. A “non-coupled” or “free” PEG molecule, as used herein, refers to a PEG molecule that is not covalently bound to another molecule. However, such PEG molecule may interact with other molecules via non-covalent interaction such as ionic interaction, hydrophobic interaction, hydrogen bonds, van der Waals forces etc.

In preferred embodiments, the at least one lipid bilayer further comprises a molecule that target the nanoliposome to foam cells. As used herein, the term “foam cell” refers to a cell which has been stimulated by a foam cell stimulating ligand to have an enhanced ability to take up lipoproteins in comparison with a cell which has not been so stimulated. Enhanced uptake may be measured according to conventional procedures known in the art. Foam cells can be identified morphologically as well. Once they have taken up lipid, they appear larger than a normal macrophage, but smaller than a giant cell. They appear to lack interdigitation pseudopodia. They are lipid-laden, loaded with droplets of lipid to the apparent visual exclusion of reticulum and organelles. The droplets are approximately one tenth the size of the nucleus.

The term “molecule targeting the nanoliposome to foam cells” or “targeting molecule”, as interchangeably used herein, includes molecules that contain at least one binding site that specifically binds to a structure or binding partner located in foam cells. By “specifically binds” it is meant that the binding molecules exhibit essentially background binding to the binding molecule or structure. The term “specificity”, as used herein, refers to the ability of a binding moiety to bind preferentially to one binding molecule, versus a different antigen, and does not necessarily imply high affinity (as defined further herein). A binding moiety that can specifically bind to and/or that has affinity for a specific binding molecule is said to be “against” or “directed against” said antigen or antigenic determinant. A targeting molecule according to the invention is said to be “cross-reactive” for two different analyte molecules if it is specific for both these different analyte molecules. The term “affinity”, as used herein, refers to the degree to which a binding molecule binds to an analyte molecule so as to shift the equilibrium of free analyte molecule and binding molecule toward the presence of a complex formed by their binding. Thus, for example, where a targeting molecule and binding molecule are combined in relatively equal concentration, a targeting molecule of high affinity will bind to the available binding molecule so as to shift the equilibrium toward high concentration of the resulting complex. The dissociation constant (K_{d}) is commonly used to describe the affinity between the binding molecule and the its target. Typically, the dissociation constant is lower than 10⁵ M. Preferably, the dissociation constant is lower than 10⁶ M, more preferably, lower than 10⁷ M. Most preferably, the dissociation constant is lower than 10⁸ M.

The terms “specifically bind” and “specific binding”, as used herein, generally refers to the ability of a targeting molecule to preferentially bind to a particular biding molecule that is present in a foam cell. In certain embodiments, a specific binding interaction will discriminate between desirable and undesirable molecules in the cell, in some embodiments more than about 10 to 100-fold or more (e.g., more than about 1000- or 10,000-fold).

In various embodiments, the targeting molecule is selected from the group consisting of protein, preferably antibody, nucleotide and natural ligand. The term “protein”, as used herein, relates to one or more polypeptides, wherein the polypeptides consist of amino acids coupled by peptide (amide) bonds. The term polypeptide refers to a polymeric compound comprised of covalently linked amino acid residues. The amino acids are preferably the 20 naturally occurring amino acids glycine, alanine, valine, leucine, isoleucine, phenylalanine, cysteine, methionine, proline, serine, threonine, glutamine, asparagine, aspartic acid, glutamic acid, histidine, lysine, arginine, tyrosine and tryptophan.

As used herein, the term “antibody” refers to an intact immunoglobulin including monoclonal antibodies, such as chimeric, humanized or human monoclonal antibodies, or to an antigen-binding and/or variable domain comprising fragment of an immunoglobulin that competes with the intact immunoglobulin for specific binding to the binding partner of the immunoglobulin. Regardless of structure, the antigen-binding fragment binds with the same antigen that is recognized by the intact immunoglobulin. The term “antibody”, as used herein, includes immunoglobulins from classes and subclasses of intact antibodies. These include IgA, IgD, IgE, IgG, and IgM, and several of these may be further divided into subclasses (isotypes), e.g., IgA1, IgA2, IgG1, IgG2, IgG3 and IgG4 as well as antigen-binding fragments thereof.

Antigen-binding fragments include, inter alia, Fab, F(ab′), F(ab′)2, Fv, dAb, Fd, complementarity determining region (CDR) fragments, single-chain antibodies (scFv), bivalent single-chain antibodies, diabodies, triabodies, tetrabodies, (poly)peptides that contain at least a fragment of an immunoglobulin that is sufficient to confer specific antigen binding to the (poly)peptide, etc. The above fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, edited by E. Harlow and D. Lane (1988), Cold Spring Harbor Laboratory, Cold Spring Harbor, N.Y., which is incorporated herein by reference. A binding molecule or antigen-binding fragment thereof may have one or more binding sites. If there is more than one binding site, the binding sites may be identical to one another or they may be different.

The term “nucleotide”, as used herein, refers to ribonucleotides, deoxyribonucleotides, dideoxynucleotides, acyclic derivatives of nucleotides, and functional equivalents thereof, of any phosphorylation state. Functional equivalents of nucleotides are those that may be functionally substituted for any of the standard ribonucleotides or deoxyribonucleotides in a polymerase or other enzymatic reaction as, for example, in an amplification or primer extension method. Functional equivalents of nucleotides are also those that may be formed into a polynucleotide that retains the ability to hybridize in a sequence specific manner to a target polynucleotide.

The term “ligand”, as used herein, refers to a molecule or more generally to a compound which is capable of binding to a target protein. A target protein may have a co-factor or physiological substrate bound thereto. The ligand of interest may bind elsewhere on the protein or may compete for binding e.g. with a physiological ligand. Ligands of interest may be drugs or drug candidates or naturally occurring binding partners, physiological substrates etc. Thus, the ligand can bind to the target to form a larger complex. The ligand can bind to the target with any affinity i.e. with high or low affinity. Generally, a ligand which binds to the target with high affinity may result in a more thermally stable target compared to a ligand which binds to the target with a lower affinity. Typically, a ligand capable of binding to a target may result in the thermal stabilization of that target protein by at least 0.25 or 0.5° C. and preferably at least 1, 1.5 or 2° C.

In preferred embodiments, the targeting molecule is folic acid and/or the Lyp-1 peptide according to SEQ ID NO:1. The term “folic acid” is used herein as a collective term for naturally occurring or synthetic compounds which comprise a pteridine ring, p-aminobenzoic acid and one or more glutamic acid residues. The term “folic acid”, as used herein, also encompasses biologically active forms of these compounds such as tetrahydrofolic acid.

The term “Lyp-1 peptide”, as used herein, refers to a peptide having the amino acid sequence of H-Cys-Gly-Asn-Lys-Arg-Thr-Arg-Gly-Cys-OH (SEQ ID NO:1). In some embodiments the Cys residues may be linked by a disulfide bond (S—S bonded). In other embodiments, the Cys residues are not linked or are linked to each other after the incorporation into the nanoliposome by changing the redox conditions of the environment to which the nanoliposome is exposed.

In a second aspect, the present invention is directed to the nanoliposome of the present invention for use as a medicament.

In a further aspect, the invention relates to the nanoliposome of the invention for use in the treatment of a cardiovascular disease. The term “cardiovascular disease”, as used herein, is intended to refer to all pathological states leading to a narrowing and/or occlusion of blood vessels, including arteries, veins, arterioles, venules, and capillaries, throughout the body. In particular, the term “cardiovascular disease” refers to conditions including atherosclerosis, thrombosis and other related pathological states, especially within arteries of the heart and brain. Accordingly, the term “cardiovascular disease” encompasses, without limitation, various types of heart disease, as well as Alzheimer's disease, vascular dimension, arteriolosclerosis, hyperlipidemic syndrome, coronory spasm, congestive heart failure (HF), coronary artery disease (CAD), arrhythmia, pericarditis, and acme myocardial infarction (MI). In preferred embodiments of the invention, the cardiovascular disease is atherosclerosis.

“Atherosclerosis”, as used herein, refers to a form of vascular disease characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease, including restenosis following revascularization procedures, coronary heart disease (also known as coronary artery disease or ischemic heart disease), cerebrovascular disease including multi-infarct dementia, and peripheral vessel disease including erectile dysfunction, are all clinical manifestations of atherosclerosis and are therefore encompassed by the terms “atherosclerosis” and “atherosclerotic disease”.

In preferred embodiments, the treatment of atherosclerosis includes the treatment of middle to late stage atherosclerotic plaques. The term “atherosclerotic plaque”, as used herein, refers to a structure build up inside the arteries. A plaque is made of cholesterol, fatty substances, cellular waste products, calcium and fibrin and is clotting the blood vessel. Atherosclerosis is divided into three sub-stages. The first (early) stage of atherosclerosis is the formation of the fatty streak on the endothelial lining (inner layer) of the arteries. The formation of a fibrous plaque in the inner wall of the arteries is the second (mid) stage of atherosclerosis. The plaque consists also of a huge number of macrophages, smooth muscle cells and lymphocytes. The final (late) stage of atherosclerosis will start when a fibrous plaque ruptures, revealing the cholesterol and connective tissue layer under it, thus provoking an intense blood coagulation reaction (leading to the formation of multiple blood clots or thrombi).

Finally, the present invention relates in a fourth aspect to a method to prepare the nanoliposome of the invention, comprising: a) providing a composition comprising the lipids forming the at least one lipid bilayer and a solvent; b) adding the at least one corticosteroid to the composition of step a); and c) removing the solvent to prepare the nanoliposome of the invention. The terms “remove” or “substantially remove”, as used herein, mean to remove at least 10%, more preferably at least 50%, and still more preferably at least 80% or at least 90% or at least 95% of the solvent from the composition to prepare the nanoliposome of the invention.

The term “solvent”, as used herein, refers to a fluid that has at least one non-aqueous fluid. Examples of suitable candidates for non-aqueous fluids that may be used include but not limited to C₁ to C₃₀ hydrocarbons, and combinations thereof, and more preferably to C₁ to C₅ hydrocarbons. The preferred hydrocarbons herein include C₁-C₃. Examples of suitable hydrocarbons include but not limited to methanol, ethanol, isopropanol, pentanes, hexanes, heptanes, octanes, nonanes, decanes, undecanes, dodecanes, tridecanes, tetradecanes, linear and cyclic paraffins, diluent, kerosene, light and heavy naphtha and combinations thereof. The term “solvent”, as used herein, also refers to compositions of different fluids.

In preferred embodiments, said method further comprises after the removing of the solvent an extruding step. Such extruding steps are well-known to the skilled person (Hope, M. J. et al., Liposome Technology, Chapter 8, REDUCTION OF LIPOSOME SIZE AND PREPARATION OF UNILAMELLAR VESICLES BY EXTRUSION TECHNIQUES, Volume I, 1993, pages 123-139, CRC Press, Inc.).

### EXAMPLES

### Example 1

### Fluocinolone Acetonide (FA)

This is the first study showing encapsulation of fluocinolone acetonide in high loading concentrations into nanoliposomes. Different drug/lipid mole ratios (D/L ratios up to 0.2) were tested in plain and pegylated liposomes comprising of saturated and unsaturated lipids.

### Sample 1: For Drug/Lipid Ratio of 0.15

### Preparation of Large Unilamellar Vesicles (LUVs)

The liposomal formulations were prepared by thin film hydration technique. DPPC and DSPE-PEG 2K were weighed and dissolved in chloroform:methanol (2:1 v/v) solvent mixture in a round bottom flask. To this lipid solvent mixture, fluocinolone acetonide was added at a drug:lipid mole ratio of 0.15:1. The solvent mixture was removed by using a rotary evaporator connected to a water bath maintained at 40° C. The flask was rotated at 150 rpm for 1 hour for thorough removal of solvents, yielding a thin drug-loaded lipid film. To this thin film, isotonic phosphate buffered saline (PBS; 150 mM, pH 7.4) was added to render the formation of multilamellar vesicles (MLVs). The MLVs were extruded through polycarbonate filters (0.2 μm for 5 times/0.08 μm for 7 times) sequentially to form LUVs with size distribution of 0.08-0.12 μm in diameter.

### Characterization of Drug-Loaded Liposomes

### Size Stability Study

The average size as well as the size distribution (polydispersity index) of the liposomes was characterized by using the Malvern Zetasizer Nano ZS. The particle sizes were measured after preparation and continuously monitored on storage (4° C.) and after drug release in vitro.

### Drug Partition Coefficient Estimation

The value for drug partition coefficient is determined by the ratio of the drug concentration associated with the liposomes to the drug concentration distributed in the aqueous continuous phase. The estimation was done before the extrusion step. Known sample volumes of MLVs were collected in micro-centrifuge tubes and centrifuged at 13000 rpm for 20 minutes. The MLVs, due to their large sizes, were separated out from the clear supernatant. Drug concentration was estimated from the supernatant as a measure of the liposome-unassociated drug concentration. This value, when subtracted from the total drug concentration, yields liposomes-associated drug concentration. Total drug concentration was estimated by mixing a known volume of liposomes with isopropyl alcohol at a volume ratio of 1:4.

### Drug Release Study

Drug-loaded liposomal suspension of 1 mL in volume was placed in a cellulose ester dialysis bag (100 kDa MWCO, 1.6 cm dia×6 cm length) and dialyzed against 40 mL of PBS pH 7.4. The dialysis was carried out on an orbital shaker run at 50 rpm inside an incubator maintained at 37° C. Aliquots were withdrawn every 24 hours from the release medium and assayed for the released drug. The release medium was exchanged completely every 24 hours with fresh PBS pH 7.4 to maintain dynamic sink condition.

Estimation of Drug Concentration

The concentration of fluocinolone acetonide was estimated using UV/Vis spectrophotometer (Tecan, infinite M200) at wavelength of 243 nm. A sample volume of 150 μL was used in a 96-well microplate (Costar 3635). The drug estimation was compared with a standard calibration curve of fluocinolone acetonide in PBS pH 7.4.

### Results

### Drug Partition Coefficient Value and Drug Loading Efficiency

A partition coefficient value of 5±1 was estimated for both DPPC and 5 mol % DSPE-PEG 2K incorporated DPPC multilamellar liposomal formulations. This value translates to 80-85% of the drug associated with the liposomes. High loading efficiency was achieved at (93±10) % for both formulations with an initial drug to lipid mole ratio of 0.15. Loading efficiency indicates the percentage of drug remains in the liposomal system after extrusion. The final fluocinolone acetonide concentration in both formulations after extrusion was estimated to be around 1 mg/mL. The final drug/lipid mole ratio value was estimated to be 0.138±0.015.

### In-Vitro Size Stability

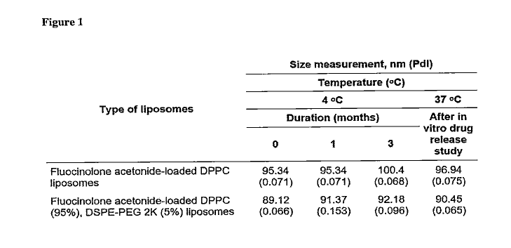

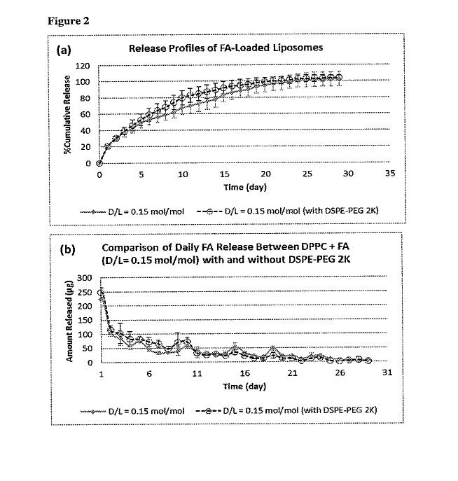

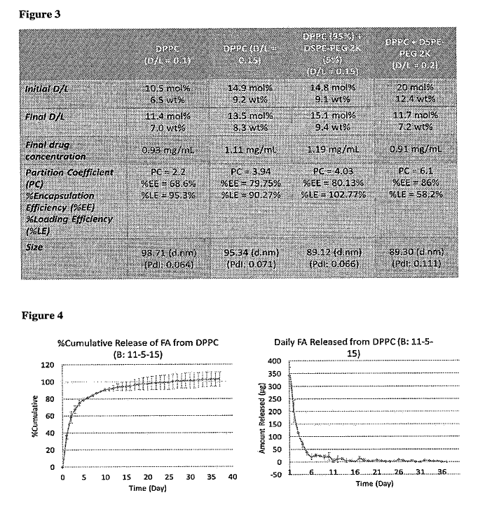

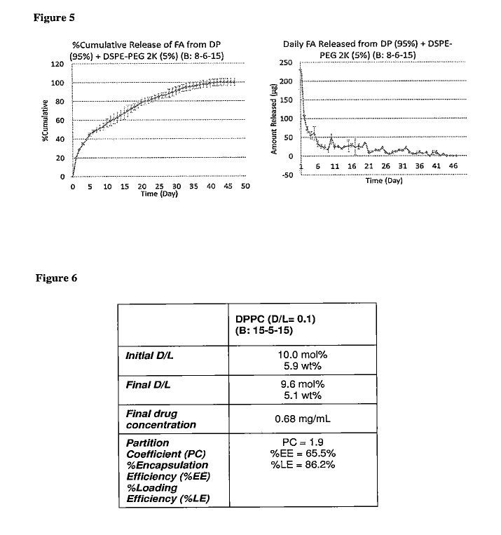

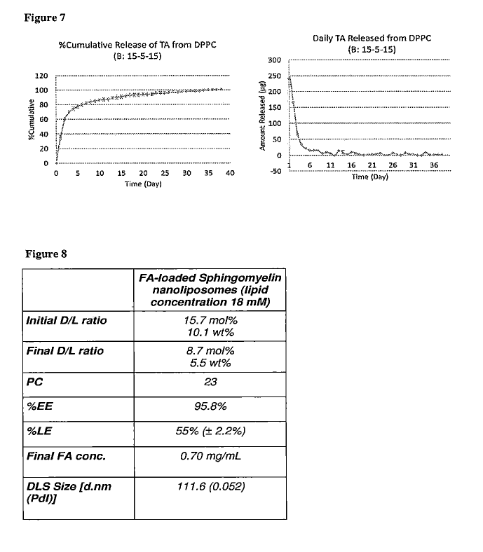

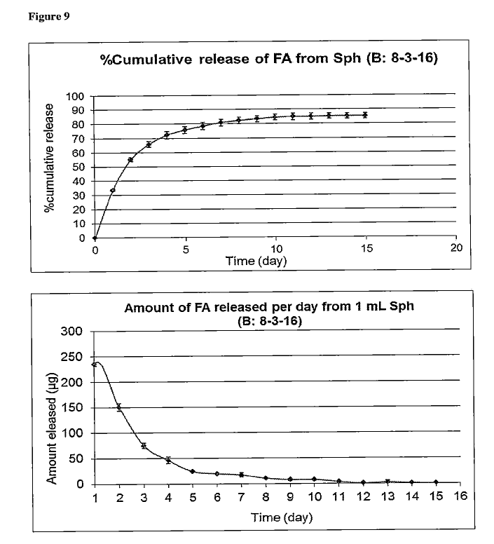

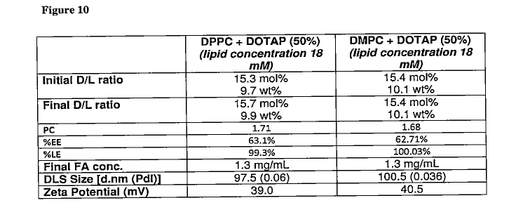

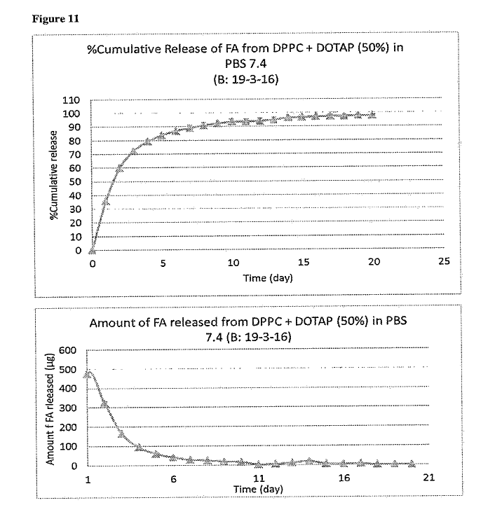

The changes in the size of the liposomes upon storage as well as during drug release study were continuously monitored with a Zetasizer (Malvern Instruments, Malvern, UK). As shown in FIG. 1, both formulations of fluocinolone acetonide-loaded liposomes were stable for at least 3 months in storage at 4° C. Particles sizes were also stable throughout the duration of release study at 37° C.

### In-Vitro Drug Release Study

The release of fluocinolone acetonide from the liposomes was evaluated by a dialysis technique and expressed in terms of cumulative drug release (%) over time as shown in FIG. 2. The release of FA from the liposomes was sustained for up to 30 days in-vitro. Incorporation of 5 mol % DSPE-PEG 2K did not show any significant effect on the release behavior and similar to plain DPPC liposomes. In addition, the vesicle size changes were minimal at the end of the in-vitro drug release study period. The results are summarized in FIG. 3.

### Sample 2: For Drug/Lipid Ratio of 0.10

The experimental procedures are similar to the methods described in sample 1. The release behaviors and amount released per day are shown in FIG. 4.

### Sample 3: For Drug/Lipid Ratio of 0.20

The experimental procedures are similar to the methods described in sample 1. The release behaviors and amount released per day are shown in FIG. 5.

### Example 2

### Triamcinolone Acetonide (TA)

Sustained release was also demonstrated with another corticosteroid drug triamcinolone acetonide (TA) from nanoliposomes, which is the first study showing high loading and sustained release of this drug from nanoliposomes. The preparation and release are exactly similar to the methods described before. Results of loading and release of TA from pegylated liposomes are shown in FIGS. 6 and 7, respectively.

### Example 3

### Nanoliposomes Comprising of Sphingolipids

The experimental procedures are similar to the methods described previously. Initial drug/lipid mole ratio of 0.15 was tested. Results of loading and release of FA from nanoliposomes comprising sphingolipids are shown in FIGS. 8 and 9, respectively.

### Example 4

### Nanoliposomes Comprising of Charged Lipids

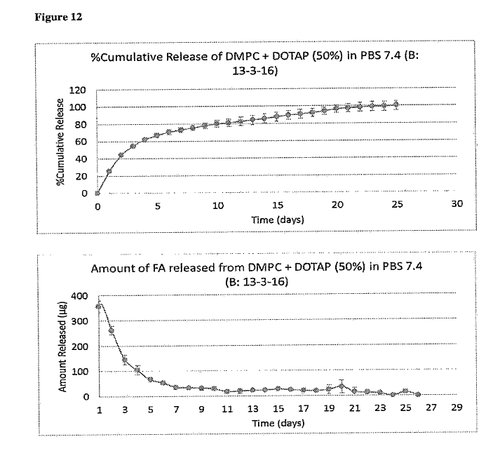

The experimental procedures are similar to the methods described previously. The sustained release of Fluocinolone Acetonide (FA) from DPPC- and DMPC-based charged nanoliposomes has been demonstrated. The results of the drug loading and release are shown in FIGS. 10-12.

### Example 5

### Nanoliposomes with Higher Lipid Concentration of 36 mM

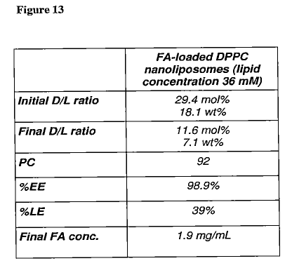

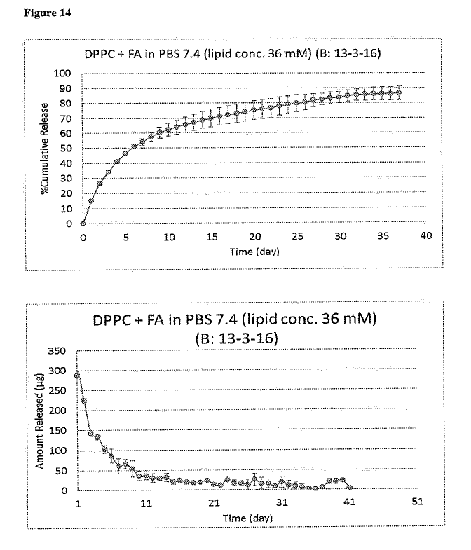

The sustained release of Fluocinolone Acetonide (FA) from DPPC nanoliposomes with lipid concentration twice as high as previously tested (36 mM vs 18 mM) has been demonstrated. The experimental procedures are exactly similar to the methods described previously. Initial drug/lipid mole ratio of 0.3 was tested. Shown in FIGS. 13 and 14 are the results of its FA loading and release.

The invention has been described broadly and generically herein. Each of the narrower species and subgeneric groupings falling within the generic disclosure also form part of the invention. This includes the generic description of the invention with a proviso or negative limitation removing any subject-matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

1. A nanoliposome comprising at least one outer lipid bilayer and at least one corticosteroid encapsulated by the at least one lipid bilayer.

2. The nanoliposome according to claim 1, wherein the ratio of the at least one corticosteroid and the lipids forming the at least one lipid bilayer is between 0.01 and 0.5.

3. The nanoliposome according to claim 1, wherein the ratio of the at least one corticosteroid and the lipids forming the at least one lipid bilayer is between 0.1 and 0.3.

4. The nanoliposome according to claim 1, wherein the ratio of the at least one corticosteroid and the lipids forming the at least one lipid bilayer is between 0.12 and 0.18.

5. The nanoliposome according to claim 1, wherein the size of the liposome is between 10 nm to 1000 nm, preferably between 50 nm to 150 nm.

6. The nanoliposome according to claim 1, wherein the at least one lipid bilayer comprises at least two different types of lipids.

7. The nanoliposome according to claim 1, wherein the lipids forming the at least one lipid bilayer are modified by polyethylene glycol (PEG) and/or the at least one lipid bilayer comprises non-coupled polyethylene glycol (PEG).

8. The nanoliposome according to claim 1, wherein lipids of the at least one lipid bilayer are selected from the group consisting of phosphocholines and sphingolipids.

9. The nanoliposome according to claim 1, wherein lipids of the at least one lipid bilayer are selected from the group consisting of dipalmitoylphosphatidylcholin (DPPC), 1,2-distearoyl-sn-glycero-3-phosphoethanolamine (DSPE), sphingomyelin, N-[1-(2,3-Dioleoyloxy)propyl]-N,N,N-trimethylammonium methyl-sulfate (DOTAP), 1,2-dimyristoyl-sn-glycero-3-phosphocholine (DMPC), 1,2-dimyristoyl-sn-glycero-3-phospho-(1′-rac-glycerol) (DMPG), soy hydrogenated L-α-phosphatidylcholine (HSPC), cholesterol, 1,2-distearoyl-sn-glycero-3-phospho-(1′-rac-glycerol) (DSPG), 1,2-dioleoyl-sn-glycero-3-phosphocholine (DOPC), 1,2-dipalmitoyl-sn-glycero-3-phospho-(1′-rac-glycerol) (DPPG), 1,2-distearoyl-sn-glycero-3-phosphocholine (DSPC), phospholipids from hen's egg, soybean oil and polysorbate 80.

10. The nanoliposome according to claim 1, wherein the corticosteroid is a group B corticosteroid.

11. The nanoliposome according to claim 1, wherein the corticosteroid is selected from the group consisting of triamcinolone acetonide, fluocinolone acetonide, triamcinolone alcohol, mometasone, amcinonide, budesonide, desonide, fluocinonide and halcinonide.

12. The nanoliposome according to claim 1, wherein the at least one lipid bilayer further comprises a molecule that target the nanoliposome to foam cells.

13. The nanoliposome according to claim 12, wherein the targeting molecule is folic acid and/or the Lyp-1 peptide according to SEQ ID NO:1.

14. (canceled)

15. A method of treating a cardiovascular disease in a subject, the method comprising administering a therapeutic amount of a nanoliposome according to claim 1 to a subject in need thereof.

16. The method according to claim 15, wherein the cardiovascular disease is atherosclerosis.

17. The method according to claim 16, wherein the treatment of atherosclerosis includes the treatment of middle to late stage atherosclerotic plaques.

18. A method to prepare the nanoliposome according to claim 1, the method comprising:
a) providing a composition comprising the lipids forming the at least one lipid bilayer and a solvent;
b) adding the at least one corticosteroid to the composition of step a); and
c) removing the solvent to prepare the nanoliposome according to claim 1.

19. The method according to claim 18, wherein said method after the removing of the solvent further comprises an extruding step.


SEQUENCE LISTING (1 sequence)

SEQ ID NO: 1 - protein, 9 aa, Artificial Sequence
  Lyp-1 peptide
CGNKRTRGC